(19) United States
(12) Patent Application Publication
(10) Pub. No.: US 2023/0390276 A1
(43) Pub. Date: Dec. 7, 2023

(54) TREATMENT OF AUTOIMMUNE DISEASES WITH A DIHYDROOROTATE HEHYDROGENASE (DHODH) INHIBITOR

(71) Applicants: Aslan Pharmaceuticals Pte. Ltd., Singapore (SG); ALMIRALL, S.A., Barcelona (ES)

(72) Inventors: Robert MOORE, Singapore (SG); Isana ENDO, Singapore (SG); Carl FIRTH, Singapore (SG); Stephen DOYLE, Singapore (SG); Nuria GODESSART MARINA, Barcelona (ES)

(21) Appl. No.: 18/249,224

(22) PCT Filed: Oct. 15, 2021
(86) PCT No.: PCT/SG2021/050625

§ 371 Date: Apr. 14, 2023

(30) Foreign Application Priority Data
Oct. 15, 2020 (SG) 10202010254Q
Dec. 21, 2020 (SG) 10202012817S

(51) Int. Cl. A61K 31/455; A61K 45/06; A61P 37/00; A61P 19/02; A61P 25/02

(52) U.S. Cl. CPC A61K 31/455; A61K 45/06; A61P 37/00; A61P 19/02; A61P 25/02

(57) ABSTRACT

A method of treating an autoimmune disease in a patient comprising administering a therapeutically effective amount of a DHODH inhibitor or a pharmaceutically acceptable salt thereof. Also provided are formulations or compositions suitable for treating autoimmune diseases.

The present disclosure relates to use of a DHODH inhibitor for the treatment of an autoimmune disease, alone or in combination with another therapy.

### BACKGROUND

Autoimmune disease pathology is characterised the body attacking itself with aberrant T cells and/or B cells. Whilst ‘autoimmune diseases’ covers a number of specific diseases, there are some fundamentally similar underlying mechanisms driving the biological processes. Common symptoms of many autoimmune diseases include: fatigue, joint pain and swelling, skin problems, abnormal pain or digestive issues, recurring fever, swollen glands.

The risk factors for autoimmune disease include genetics (particularly multiple sclerosis (MS)), obesity, smoking and certain medications such as antibiotics, statins and some medications used to lower blood pressure.

The resulting disease is difficult to diagnose, treat and can be very debilitating. There is a real unmet need for patients with autoimmune disease. In some instances when MS is really severe the approach is to wipe the patient's immune system and perform a stem cell transplant. This is a dangerous, difficult and expensive procedure and is a treatment of last resort.

Purine and pyrimidine nucleotides play critical roles in DNA and RNA synthesis as well as in membrane lipid biosynthesis and protein glycosylation. They are necessary for the development and survival of mature T lymphocytes. Activation of T lymphocytes is associated with an increase of purine and pyrimidine pools, which in turn leads to a marked increase in activity of key enzymes involved in de novo purine and pyrimidine synthesis.

Pyrimidine is believed to be important for controlling progression from early to intermediate S phase of T cell life cycle. Inhibition of pyrimidine also causes apoptosis of activated T cells.

Similarly, biosynthesis of pyrimidine is also important in the life cycle of activated B cells.

Whilst not wishing to be bound by theory the present inventors believe that blockade of the biosynthesis of pyrimidine causes cell cycle arrest and/or apoptosis in activated T cells and/or B cells with aberrant activity in autoimmune disease.

Dihydroorotate dehydrogenase (DHODH) is the enzyme that catalyzes the fourth step in the pyrimidine biosynthetic pathway namely the conversion of dihydroorotate to orotate concomitantly with an electron transfer to ubiquinone (cofactor Q) via a flavin mononucleotide intermediate (Loftier MoI Cell Biochem, 1997). In contrast to parasites (Plasmodium falciparum) (McRobert et al MoI Biochem Parasitol 2002) and bacteria (E. coli) which exclusively have this de novo pathway as the source of pyrimidines, mammal cells have an additional salvage pathway.

During homeostatic proliferation the salvage pathway, which is independent of DHODH, seems sufficient for the cellular supply with pyrimidine bases. However, in cells with a high turnover the de novo pathway is required to proliferate. In these cells, DHODH inhibition stops the cell cycle progression by suppressing DNA synthesis and ultimately cell proliferation (Breedveld F. C. Ann Rheum Dis 2000).

There are some suggestions that inhibition of mitochondrial cytochrome bc1, a component of the electron transport chain complex III, leads to activation of p53, followed by apoptosis induction. The mitochondrial respiratory chain is coupled to the de novo pyrimidine biosynthesis pathway via the mitochondrial enzyme dihydroorotate dehydrogenase (DHODH).

The drug profile of each DHODH inhibitor is very different, for example side effects of leflunomide include arterial hypertension, myelosuppression, nausea and hair loss. Brequinar is generally employed only as a model compound because clinical trials suggest the molecule lacks the requisite activity in vivo. Vidofludimus is a next generation DHODH inhibitor, which inhibits production of proinflammatory cytokines (such as IL-17) from activated lymphocytes. However, the latter is thought to be independent of DHODH activity.

Whilst DHODH inhibitors have been of interest as therapeutics, it has been been difficult to find molecules that balance all the requisite criteria, to provide a therapeutic that is effective in vivo.

The present disclosure provides use of a specific DHODH inhibitor ASLAN003 in the treatment of an autoimmune disease, such as multiple sclerosis, autoimmune skin disease and inflammatory bowel disease (for example Crohn's).

ASLAN003 has activity against the underlying cause of autoimmune diseases, namely aberrant T cells and/or B cells, which surprisingly translates in vivo to the broad-spectrum activity against autoimmune disease wherein the off-target effects are minimal, in particular liver toxicity. ASLAN003 has high affinity for DHODH and is effective in the clinic. ASLAN003 is a next generation DHODH inhibitor which is well tolerated and delivers excellent results to patients. It has the ability to positively impact on patient quality of life to control disease status, and to halt its progression and/or put the disease into remission. Advantageously, healthy cells which have a lower metabolic burden are generally unaffected by the treatment. The balanced characteristics of the treatment are extremely beneficial to patients.

Whilst not wishing to be bound by theory, it is believed that in one embodiment the present treatment is disease modifying. That is after a period of treatment the body is able to reset itself and send the autoimmune disease into remission, for example without the need to continue administering the therapy or where the therapy is continued at a low dose for maintenance.

### SUMMARY OF THE DISCLOSURE

The present disclosure is summarised in the following paragraphs:
1. A method of treating an autoimmune disease in a patient comprising administering a therapeutically effective amount of a DHODH inhibitor 2-(3,5-difluoro-3′methoxybiphenyl-4-ylamino)nicotinic acid or a pharmaceutically acceptable salt thereof
1A A DHODH inhibitor 2-(3,5-difluoro-3′methoxybiphenyl-4-ylamino)nicotinic acid or a pharmaceutically acceptable salt thereof for use in a patient with or suspected of having an autoimmune disease.
1B Use of a DHODH inhibitor 2-(3,5-difluoro-3′methoxybiphenyl-4-ylamino)nicotinic acid or a pharmaceutically acceptable salt thereof in the manufacture of a medicament for the treatment of an autoimmune disease.
2. A method, inhibitor or use according to paragraph 1, 1A or 1B, wherein the autoimmune disease is selected from the group comprising or consisting of Hidradenitis suppurativa, Scleroderma (Systemic scleritis), Lichen planus, Morphea, Psoriasis, Diabetes mellitus type 1, Autoimmune thyroiditis, Graves' disease, Endometriosis, Coeliac disease, Crohn's disease, Ulcerative colitis, Axial spondylitis, Juvenile arthritis, Palindromic rheumatism, Psoriatic arthritis, Rheumatoid arthritis, Sarcoidosis, Systemic lupus erythematosus (SLE), Undifferentiated connective tissue disease (UCTD), Multiple sclerosis, pattern II, Restless legs syndrome, Optic neuritis, Uveitis, Scleritis, Mooren's ulcer, Ménière's disease, Graves' ophthalmopathy, Neuromyelitis optica, Susac's syndrome, and lupus erythematosus.
3. A method, inhibitor or use according to paragraph 1, 1A or 1B, wherein the autoimmune disease is inflammatory bowel disease, for example Coeliac disease, Crohn's disease, Ulcerative colitis, such as Crohn's.
4. A method, inhibitor or use according to paragraph 1, 1A or 1B, wherein the autoimmune disease is a skin disorder.
5. A method, inhibitor or use according to any one of paragraphs 1, 1A, or 1B to 4, wherein the autoimmune disease is characterised by aberrant T cell responses.
6. A method, inhibitor or use according to any one of paragraphs 1, 1A, or 1B to 5, wherein the autoimmune disease is characterised by aberrant B cell responses.
7. A method, inhibitor or use according to any one of paragraphs 1, 1A, or 1B to 6, wherein the autoimmune disease is inadequately controlled, for example by standard of care medicine.
8. A method, inhibitor or use according to any one of paragraphs 1, 1A, or 1B to 7, wherein the autoimmune disease is severe.
9. A method, inhibitor or use according to any one of paragraphs 1, 1A, or 1B to 8, wherein the autoimmune disease is not one or more of the following: rheumatoid arthritis, psoriatic arthritis, ankylosing spondilytis, multiple sclerosis, Wegener's granulomatosis, systemic lupus erythematosus, psoriasis, sarcoidosis polyarticular juvenile idiopathic arthritis, inflammatory bowel disease (such as ulcerative colitis and Crohn's disease), Reiter's syndrome, fibromyalgia or type-1 diabetes.
10. A method, inhibitor or use according to any one of paragraphs 1, 1A, or 1B to 8, wherein the autoimmune disease is inflammatory bowel disease, for example colitis including ulcerative colititis, Crohn's disease and/or coeliac disease (and complications thereof, such as arthritis, uveitis, erythema nodosum and jaundice)
11. A method, inhibitor or use according to any one of paragraphs 1, 1A, or 1B to 8, wherein the autoimmune disease is a skin disorder, for example atopic dermatitis (including autoimmune progesterone dermatitis), psoriasis, erythema (including erythema nodosum), scleroderma, and lupus.
12. A method, inhibitor or use according to any one of claims 1, 1A, or 1B to 8, wherein the autoimmune disease is rheumatoid arthritis.
13. A method, inhibitor or use according to any one of paragraphs 1, 1A, or 1B to 8, wherein the autoimmune disease is multiple sclerosis.
14. A method, inhibitor or use according to paragraph 13, wherein the multiple sclerosis is relapsing remitting multiple sclerosis.
15. A method, inhibitor or use according to paragraphs 13 or 14, wherein the multiple sclerosis is secondary progressive multiple sclerosis.
16. A method, inhibitor or use according to paragraph 13, wherein the multiple sclerosis is primary progressive multiple sclerosis.
17. A method, inhibitor or use according to any one of paragraphs 13 to 16, wherein the severity of symptoms is reduced, for example motor function, spasticity, muscle spasms and/or stiffness is improved with treatment.
18. A method, inhibitor or use according to any one of paragraphs 13 to 17, wherein frequency or severity of relapses are reduced, with treatment
19. A method, inhibitor or use according to any one of paragraphs 1 to 18, wherein aberrant T cell activation is minimised, for example progression from early to intermediate S phase of T cell life cycle is inhibited.
20. A method, inhibitor or use according to paragraph 19, wherein there is decreased activation of memory T cells.
21. A method, inhibitor or use according to paragraph 19 or 20, wherein aberrant activation of TH1 T cells is inhibited.
22. A method according to any one of paragraphs 1, 1A, or 1B to 21, wherein TH2 T cell activation is not inhibited.
23. A method, inhibitor or use according to any one of paragraphs 1, 1A, or 1B to 22, wherein aberrant B cell activation is inhibited, for example through limiting progression of S phase in the cell life cycle is inhibited.
24. A method, inhibitor or use according to any one of paragraphs 1, 1A, or 1B to 23, wherein apoptosis of aberrant immune cells in stimulated.
25. A method, inhibitor or use according to any one of paragraphs 1, 1A, or 1B to 24, wherein the treatment is for a flare-up of the autoimmune disease.
26. A method, inhibitor or use according to any one of paragraphs 1, 1A, or 1B to 25, wherein the treatment is continuous, for example 100 to 400 mg per dose (such as per day), in particular 300 to 400 mg per dose (such as per day) for at least a period and optionally a maintenance for example 100 to 200 mg per dose (such as per day).
27. A method, inhibitor or use according to any one of paragraphs 1, 1A, or 1B to 25, wherein the treatment is intermittent or is discontinued after a period, for example after a defined endpoint.
28. A method, inhibitor or use according to any one of paragraphs 1, 1A, or 1B to 27, wherein the DHODH inhibitor is employed as monotherapy.
29. A method, inhibitor or use according to any one of paragraphs 1, 1A, or 1B to 27, wherein the DHODH inhibitor is employed in a combination therapy.
30. A method, inhibitor or use according to paragraph 29, wherein the combination therapy comprises a treatment independently selected from corticosteroids (for example oral prednisone and intravenous methylprednisolone), plasma exchange (plasmapheresis), interferon beta medications, glatiramer acetate, fingolimod, dimethyl fumarate, diroximel fumarate, teriflunomide, siponimod, cladribine, ocrelizumab, natalizumab, an anti-CD20 agent or biosimilar thereof, such as rituximab, alemtuzumab, and a Bruton's Tyrosine Kinase (BTK) inhibitor.
31. A method, inhibitor or use according to paragraphs 29 or 30, wherein the combination therapy comprises a treatment to ease or reduce the symptoms of multiple sclerosis, for example a muscle relaxant (such as baclofen, tizanidine and cyclobenzaprine), a medication to reduce fatigue (such as amantadine, modafinil and methylphenidate), and/or a medication to increase walking speed (such as dalfampridine).
32. A method, inhibitor or use according to any one of paragraphs 29 to 31, wherein the combination therapy comprises an antidepressant, for example a tricyclic antidepressant, such as clomipramine.
33. A method, inhibitor or use according to any one of paragraphs 29 to 32, wherein the combination therapy comprises duloxetine.
34. A method, inhibitor or use according to any one of paragraphs 29 to 33, wherein the combination comprises mirabegron and/or desmopressin.
35. A method, inhibitor or use according to any one of paragraphs 29 to 34, wherein the combination therapy comprises interferon beta (IFN-β) such as interferon beta-1a or interferon beta-1b.
36. A method, inhibitor or use according to any one of paragraphs 29 to 35, wherein the combination therapy comprises an anti-CD20 agent or a biosimilar thereof, for example Rituxan (rituximab), a Rituximab biosimilar, Gazyva, Kesimpta, Ocrevus (ocrelizumab), Ruxience, Truxima, Zevalin, Arzerra, AcellBia, HLX01, Reditux, Ritucad or Zytux.
37. A method, inhibitor or use according to any one of paragraphs 29 to 36, wherein the combination therapy comprises a Bruton's Tyrosine Kinase (BTK) inhibitor, for example Ibrutinib, Acalabrutinib, Zanubrutinib, Evobrutinib, ABBV-105, Fenebrutinib, GS-4059, Spebrutinib or HM71224.
38. A method, inhibitor or use according to any one of paragraphs 29 to 37, wherein the combination does not comprise methotrexate.
39. A method, inhibitor or use according to any one of paragraphs 29 to 38, wherein the combination comprises a purine synthesis inhibitor, such as azathioprine.
40. A method, inhibitor or use according to any one of paragraphs 29 to 39, wherein the combination comprises a biological therapeutic such as an antibody or binding fragment thereof in particular dupilumab or an anti-IL-13Rα1 antibody or antigen binding fragment thereof, such as ASLAN004, including a pharmaceutical formulation of any one of the same.
41. A method, inhibitor or use according to any one of paragraphs 1, 1A, or 1B to 40, wherein the DHODH inhibitor is administered at a dose in the range 1 mg to 400 mg per day, for example 100 mg to 400 mg per day, such as 100, 200, 300 or 400 mg per day.
42. A method, inhibitor or use according to any one of paragraphs 1, 1A, or 1B to 41, wherein the DHODH inhibitor is administered daily, for example once daily.
43. A method, inhibitor or use according to any one of paragraphs 1, 1A, or 1B to 42, wherein the patient is human.
44. A method, inhibitor or use according to any one of paragraphs 1, 1A, or 1B to 43, wherein the patient has an age of at least 40 years, for example at 45, at least 50, at least 55, at least 60, at least 65, at least 70, at least 75.
45. A method, inhibitor or use according to any one of paragraphs 1, 1A, or 1B to 44, wherein the patient has co-morbidities.
46. A method, inhibitor or use according to paragraph 45, wherein the co-morbidity is selected from obesity, allergy, asthma, COPD, diabetes, kidney failure, heart disease (including heart failure), cancer, dementia, liver disease, and combinations thereof.

### BRIEF SUMMARY OF THE FIGURES

FIG. 1 graph showing results of in vivo study of ASLAN003 (LAS186323) in multiple sclerosis experimental autoimmune encephalomyelitis (EAE) model. This shows a dose dependent response when ASLAN003 is administered.

FIG. 2 graph showing mean of the clinical score Area Under Curve (AUC) for each treatment group.

FIG. 3 graph showing CNS histological score.

FIG. 4 graph showing evolution of disease with time. Y axis=Clinical score, X-axis=day of treatment, V=Vehicle, F=positive control F, T=positive control T, L=ASLAN003 (LAS186323). The numbers after the initials correspond to the administered oral daily dose of the compound in mg/kg.

FIG. 5 graph showing haematological cell count. Y axis=cell count, WBC=white blood cells, RBC=red blood cells, HGB=haemoglobin, NEUT=neutrophils, LYMPH=lymphocytes. For each cell type, the bars from left to right are: Naïve, V, F-0.1, T-1, T-3, T-10, L-1, L-3, L-10 and L-15.
V=Vehicle, F=positive control, T=positive control, L=ASLAN003 (LAS186323). The numbers after the initials correspond to the administered oral daily dose of the compound in mg/kg.

FIG. 6 graph showing (AUC) score for each treatment group in rheumatoid arthritis (RA) Adjuvant-Induced Arthritis (AIA) model. T-3: positive control T at 3 mg/kg, One-way ANOVA with Bonferroni's post-test, *p<0.05 FIG. 7 graph showing X-ray score for each treatment group in RA AIA model. T-3: positive control T at 3 mg/kg.

FIG. 8A graph showing body weight for each treatment group in dextran sulfate sodium (DSS) induced inflammatory bowel disease (IBD) model*.

FIG. 8B graph showing % change in body weight for each treatment group in DSS induced IBD model*.

FIG. 9A graph showing stool consistency score for each treatment group in DSS induced IBD model*.

FIG. 9B graph showing blood stool score for each treatment group in DSS induced IBD model*.

FIG. 10 graph showing Disease Activity Index (DAI) for each treatment group in DSS induced IBD model*.

FIG. 11 graph showing results of intestinal permeability test (FITC-concentration) for each treatment group in DSS induced IBD model*.

FIG. 12A graph showing colon weight for each treatment group in DSS induced IBD model*.

FIG. 12B graph showing colon length for each treatment group in DSS induced IBD model*.

FIG. 12C graph showing colon weight/length for each treatment group in DSS induced IBD model*.

FIG. 12D graph showing spleen weight for each treatment group in DSS induced IBD model*.

FIG. 13 graph showing lipocalin in faeces data for each treatment group in DSS induced IBD model*. * Error bars indicate standard error from mean (SEM).

### DETAILED DESCRIPTION

2-(3,5-difluoro-3′-methoxybiphenyl-4-ylamino) nicotinic acid (referred to herein as ASLAN003) has the structure:

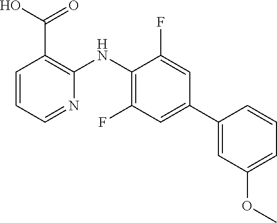 [SMILES: COc1cccc(-c2cc(F)c(Nc3ncccc3C(=O)O)c(F)c2)c1]

### Autoimmune Disease

Autoimmune disease as used herein refers to any disease or condition wherein an individual's immune system mistakenly targets that individual's own normal “healthy” cells, in particular characterised by aberrant T cell and/or B cell activation.

Aberrant T cell and/or B cell activation as employed herein refers to abnormal T cell and/or B cell activation, in particular where the abnormal cells recognise self or self antigens.

Severe autoimmune disease is where the disease is not controlled by standard of care medicaments/treatments.

Flare, is a period of disease exacerbation.

“Inadequate control” as employed herein refers to where standard of care medication fails to lessen or control symptoms, in particular where the patient's quality of life is adversely affected.

“Defined endpoint” as employed herein refers to clinically defined point, for example remission or stable disease.

In one embodiment ASLAN003 is employed in maintenance therapy, for example at a low dose. Maintenance therapy as employed herein refers to continuous therapy to make the disease stable or to keep the disease in remission, for example where the dose administered is low and in particular frequent A dose of 100 to 200 mg for example given once or twice a day may be used as maintenance therapy.

In one embodiment the autoimmune disease is an autoimmune skin disease i.e. autoimmune disease manifested or presented in the skin in particular the dermis and/or epidermis, such as lupus.

Thus, in one embodiment the autoimmune disease is selected from the group comprising or consisting of Acute disseminated encephalomyelitis (adem), acute necrotizing hemorrhagic leukoencephalitis, Addison's disease, adrenal insufficiency, hypocortisolism, amyloidosis, ankylosing spondylitis, spondyloarthritis, Strumpell-marie disease, anti-GBM/anti-TBM nephritis, antiphospholipid syndrome (aps), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune lymphoproliferative syndrome (ALPS), Canale-Smith syndrome, autoimmune myocarditis, autoimmune oophoritis, autoimmune pancreatitis (AIP), autoimmune polyglandular syndromes(types I, II & III), autoimmune retinopathy (AR), autoimmune thrombocytopenic purpura (ATP), autoimmune thyroid disease, autoimmune urticaria, axonal/neuronal neuropathies, balo disease, Behcet's disease, bullous pemphigoid, cardiomyopathy, Castleman disease, coeliac disease, chagas disease, chronic inflammatory demyelinating polyneuropathy (CIDP), chronic recurrent multifocal ostomyelitis (CRMO), Churg-Strauss syndrome, cicatricial pemphigoid/benign mucosal pemphigoid (CP), Crohn's disease, inflammatory bowel disease, colitis (including ulcerative colitis), enteritis, ileitis, Cogans syndrome, cold agglutinin disease, congenital heart block, Coxsackie myocarditis, crest disease, cryoglobulinemia, demyelinating neuropathies, dermatitis herpetiformis, Duhring's disease, dermatomyositis, diabetes, type I, discoid lupus erythematosus (DLE), Dressler's syndrome, endometriosis, epidermolysis bullosa (EB) and eb acquisita (EBA), eosinophilic gastroenteritis, esophagitis, eosinophilic fasciitis, schulman's syndrome, erythema nodosum, experimental allergic encephalomyelitis, Evans syndrome, fibrosing alveolitis, giant cell arteritis (temporal arteritis), giant cell myocarditis, glomerulonephritis (non-proliferative: focal segmental glomerulosclerosis and membranous glomerulonephritis. proliferative: IgA nephropathy), goodpasture's syndrome, granulomatosis with polyangiitis (GPA) (formerly called Wegener's granulomatosis), Graves' disease, Guillain-Barré syndrome, Miller Fisher syndrome, acute motor axonal neuropathy, acute motor sensory axonal neuropathy, acute panautonomic neuropathy, Bickerstaff's brainstem encephalitis, Hashimoto's encephalitis, Hashimoto's thyroiditis, hemolytic anemia, Henoch-Schonlein purpura, herpes gestationis, hypogammaglobulinemia, idiopathic pulmonary fibrosis, idiopathic thrombocytopenic purpura (ITP), IgA nephropathy (IGAN), berger's syndrome, synpharyngitic glomerulonephritis, IgA pemphigus, IgG4-related sclerosing disease, immune-regulated infertility, inclusion body myositis, insulin-dependent diabetes mellitus, interstitial cystitis, Isaac's syndrome, neuromyotonia, juvenile arthritis, juvenile myositis, Kawasaki syndrome, Lambert-Eaton syndrome, leukocytoclastic vasculitis, lichen planus, lichen sclerosus, ligneous conjunctivitis, linear IgA dermatosis (LAD), pemphigoid, lupus (SLE), lyme disease, Meniere's disease, microscopic polyangiitis (MPA), mixed connective tissue disease (MCTD), monoclonal gammaopathy, Mooren's ulcer, Mucha-Habermann disease, multiple sclerosis, myasthenia gravis, myositis, narcolepsy, neuromyelitis optica (devic's), neuromyotonia, Isaac's syndrome (acquired, paraneoplastic, hereditary), neutropenia, ocular cicatricial pemphigoid, optic neuritis, oophoritis, opsoclonus-myoclonus syndrome, orchitis, palindromic rheumatism, pandas (pediatric autoimmune neuropsychiatric disorders associated with streptococcus), paraneoplastic autoimmune multiorgan syndrome (PAMS), paraneoplastic cerebellar degeneration, paraneoplastic pemphigus (PNP), paroxysmal nocturnal hemoglobinuria (PNH), Parry Romberg syndrome, Parsonnage-Turner syndrome, pars planitis (peripheral uveitis), pempgigoid gestationis (PG), pemphigus vulgaris (PV), pemphigus folliaceus (PF), peripheral neuropathy, perivenous encephalomyelitis, pernicious anemia, Poems syndrome, polyarteritis nodosa (PAN), polymyalgia rheumatic, polymyositis, postmyocardial infarction syndrome, postpericardiotomy syndrome, progesterone dermatitis primary biliary cirrhosis, Hanot syndrome, primary sclerosing cholangitis (PSC), sclerosong cholangitis, psoriasis, psoriatic arthritis, pyoderma gangrenosum, pure red cell aplasia, Rasmussen's encephalitis, chronic focal encephalitis (CFE), Raynauds phenomenon, reactive arthritis, Reiter's syndrome, recoverin-associated retinopathy (RAR), reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, systemic sclerosis, Sjogren's syndrome, sperm & testicular autoimmunity, stiff person/man syndrome, subacute bacterial endocarditis (SBE), Susac's syndrome, sympathetic ophthalmia, Takayasu's arteritis, temporal arteritis/giant cell arteritis, thromboangiitis obliterans, Buerger's disease, thrombocytopenic purpura (TTP), Tolosa-Hunt syndrome, transverse myelitis, undifferentiated connective tissue disease (UCTD), uveitis, Takayasu's arteritis, temporal arteritis, Buerger's disease, cutaneous vasculitis, Kawasaki disease, polyarteritis nodosa, Behçet's syndrome, Churg-Strauss syndrome, cutaneous vasculitis, Henoch-Schönlein purpura, microscopic polyangiitis, Wegener's granulomatosis, golfer's vasculitis, vesiculobullous dermatosis, and wegener's granulomatosis (now termed granulomatosis with polyangiitis (GPA).

In one embodiment the autoimmune disease is selected from the group comprising or consisting of ANCA vasculitis, IgA nephropathy (Berger's), pemphigus vulgaris/bullous pemphigoid, ITP, primary biliary cirrhosis, autoimmune thyroiditis (Grave's disease), hashimoto's disease, lupus nephritis, membranous glomerulonephritis (or membranous nephropathy), APS, myasthenia gravis, neuromyelitis optica, primary Sjögren's, autoimmune neutropaenia, autoimmune pancreatitis, dermatosmyositis, autoimmune uveitis, autoimmune retinopathy, Behçet's disease, IPF, systemic sclerosis, liver fibrosis, autoimmune hepatitis, primary sclerosing cholangitis, goodpasture's syndrome, pulmonary alveolar proteinosis, chronic autoimmune urticarial, psoriasis, rheumatoid arthritis, psoriatic arthritis, axial spodyloarthritis, transplantation (including GvHD), asthma, COPD, giant cell arteritis, refractory autoimmune cytopaenias, Evans syndrome (autoimmune haemolytic anaemia), type I diabetes, sarcoidosis, polymyositis, ulcerative colitis, Crohn's disease, coeliac disease, Waldenstrom's macroglobulinaemia, focal segmental glomerulosclerosis, chronic Lyme disease (Lyme borreliosis), lichen planus, Stiff person syndrome, dilated cardiomyopathy, autoimmune (lymphocytic) oophoritis, epidermolysis bullosa acquisita, autoimmune atrophic gastritis, pernicious anaemia, atopic dermatitis, atherosclerosis, multiple sclerosis, Rasmussen's encephalitis, Guillain-Barré syndrome, acquired neuromyotonia, and stroke.

In one embodiment, the autoimmune disease is selected from the group comprising or consisting of Hidradenitis suppurativa, Scleroderma (Systemic scleritis), Lichen planus, Morphea, Psoriasis, Diabetes mellitus type 1, Autoimmune thyroiditis, Graves' disease, Endometriosis, Coeliac disease, Crohn's disease, Ulcerative colitis, Axial spondylitis, Juvenile arthritis, Palindromic rheumatism, Psoriatic arthritis, Rheumatoid arthritis, Sarcoidosis, Systemic lupus erythematosus (SLE), Undifferentiated connective tissue disease (UCTD), Multiple sclerosis, pattern II, Restless legs syndrome, Optic neuritis, Uveitis, Scleritis, Mooren's ulcer, Ménière's disease, Graves' opthalmopathy, Neuromyelitis optica, Susac's syndrome, and lupus erythrematosus.

In one embodiment, the autoimmune disease is selected from the group comprising or consisting of Lichen planus, Hidradenitis suppurativa, Coeliac disease, Ulcerative colitis, Crohn's disease, Graves' disease, Autoimmune thyroiditis, Endometriosis, Multiple sclerosis and Optic neuritis.

Multiple sclerosis (MS) is a disease in which the myelin sheath insulating the nerve cells in the brain and spinal cord are damaged. As a result of the damage, the ability of the nervous system to properly transmit signals is disrupted. This in turn causes a range of serious physical and mental issues, for example double vision, blindness in one eye, muscle weakness, problems with sensation or co-ordination, problems with speech, acute or chronic pain, bladder and bowel difficulties, depression, and mood swings.

The condition begins in most cases as a clinically isolated syndrome over a number of days with the majority suffering from motor or sensory problems. The course of symptoms occurs in two patterns initially, either as episodes of sudden worsening that last a few days to months, known as relapses, followed by improvement in most cases, or a gradual worsening over time without periods of recovery. Relapses are generally unpredictable and occur without warning.

The cause of multiple sclerosis is currently unclear, although the underlying mechanism is thought to be destruction of the myeline sheath by the individual's own immune system, i.e. multiple sclerosis is considered at least in part an autoimmune disease. It is the most common immune-mediated disorder affecting the central nervous system, with about 2.3 million people affected globally. However, there are a number of autoimmune diseases that have a serious and negative impact on the life of many suffers.

In one embodiment, the autoimmune disease is multiple sclerosis (MS). Multiple sclerosis is generally further classified as one of four variants: clinically isolated syndrome (CIS), relapsing-remitting MS (RRMS), primary progressive MS (PPMS) and secondary progressive MS (SPMS). Hence, in one embodiment, the autoimmune disease is selected from the group comprising CIS, RRMS, PPMS and SPMS.

Relapsing-remitting MS (RRMS) is characterized by unpredictable relapses followed by periods of months to years of relative quiet (remission) with no new signs of disease activity. The condition is typified by progressive, sustained demyelination, and associated axonal loss. In one embodiment, the autoimmune disease is RRMS.

The attacks—also called relapses or exacerbations (also referred to herein as flare)—are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

The relapsing-remitting subtype usually begins with a clinically isolated syndrome (CIS). In CIS, a person has an attack suggestive of demyelination, but does not fulfil the criteria for multiple sclerosis. 30 to 70% of persons who experience CIS, later develop MS. In one embodiment, the autoimmune disease is CIS.

Primary progressive MS (PPMS) occurs in approximately 10-20% of individuals, with no remission after the initial symptoms. It is characterized by progression of disability from onset, with no, or only occasional and minor, remissions and improvements. In one embodiment, the autoimmune disease is PPMS.

Secondary progressive MS (SPMS) occurs in around 65% of those with initial relapsing-remitting MS, who eventually have progressive neurologic decline between acute attacks without any definite periods of remission. Occasional relapses and minor remissions may appear. SPMS can be further characterized as either active (with relapses and/or evidence of new MRI activity during a specified period of time) or not active, as well as with progression (evidence of disability accumulation over time, with or without relapses or new MRI activity) or without progression. In one embodiment, the autoimmune disease is SPMS.

In one embodiment the disease is chronic inflammatory demyelinating polyneuropathy.

In one embodiment the disease is transverse myelitis.

In one embodiment the disease is neuromyelitis optica.

In one embodiment, the autoimmune disease is one or more of the following: rheumatoid arthritis, psoriatic arthritis, ankylosing spondilytis, multiple sclerosis, Wegener's granulomatosis, systemic lupus erythematosus, psoriasis, sarcoidosis, polyarticular juvenile idiopathic arthritis, inflammatory bowel disease such as ulcerative colitis and Crohn's disease, Reiter's syndrome, fibromyalgia and type-1 diabetes. In one embodiment it is not any one of the same.

In one embodiment, the autoimmune disease is psoriatic arthritis. In one embodiment it is not psoriatic arthritis.

In one embodiment, the autoimmune disease is ankylosing spondilytis. In one embodiment it is not ankylosing spondilytis.

In one embodiment, the autoimmune disease is multiple sclerosis. In one embodiment, it is not multiple sclerosis.

In one embodiment, the autoimmune disease is Wegener's granulomatosis. In one embodiment, it is not Wegener's granulomatosis.

In one embodiment, the autoimmune disease is systemic lupus erythematosus. In one embodiment it is not systemic lupus erythematosus.

In one embodiment, the autoimmune disease is psoriasis. In one embodiment it is not psoriasis.

In one embodiment, the autoimmune disease is sarcoidosis. In one embodiment, it is not sarcoidosis.

In one embodiment, the autoimmune disease is polyarticular juvenile idiopathic arthritis. In one embodiment it is not polyarticular juvenile idiopathic arthritis

In one embodiment, the autoimmune disease is an inflammatory bowel disease, such as ulcerative colitis or Crohn's disease. In one embodiment it is not an inflammatory bowel disease, such as ulcerative colitis or Crohn's disease.

In one embodiment, the autoimmune disease is Reiter's syndrome. In one embodiment it is not Reiter's syndrome.

In one embodiment, the autoimmune disease is fibromyalgia. In one embodiment it is not fibromyalgia.

In one embodiment, the autoimmune disease is type-1 diabetes. In one embodiment it is not type-1 diabetes.

In one embodiment the autoimmune disease is arthritis, such as rheumatoid arthritis. In one embodiment it is not arthritis, such as rheumatoid arthritis.

### Formulations

A DHODH inhibitor is a moiety (such as a compound) that inhibits, for example reduces or blocks the activity of a DHODH enzyme (see background for definition thereof).

In one embodiment the DHODH inhibitor is provided as a pharmaceutical formulation.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes.

In one embodiment the pharmaceutical formulation is for oral administration, for example formulated as tablets, pills, dragees, capsules, liquids, gels, syrups, slurries and suspensions, for ingestion by the patient.

Excipients may include lactose, dextrin, glucose, sucrose, sorbitol, starch, sugars, sugar alcohols and cellulose.

Other suitable forms for administration include parenteral administration, for example injection or infusion, such as bolus injection or continuous infusion.

Where the product is for injection or infusion, it may take the form of a suspension, solution or emulsion in an oily or aqueous vehicle and it may contain formulatory agents, such as a suspending agent, preservative, stabilising and/or dispersing agents. Alternatively, the molecule may be in dry form, for reconstitution before use with an appropriate sterile liquid. Pharmaceutically acceptable carriers in therapeutic compositions may additionally contain liquids such as water, saline, glycerol and ethanol. Additionally, auxiliary substances, such as wetting agent, emulsifying agents, lubricant or pH buffering substances, may be present in such compositions.

A thorough discussion of pharmaceutically acceptable carriers is available in Remington's Pharmaceutical Sciences (Mack Publishing Company, N.J. 1991).

### Treatment

Treatment as employed herein refers to where the patient has a disease or disorder, for example autoimmune disease (in particular one disclosed herein) and the medicament according to the present disclosure is administered to stabilise the disease, delay the disease, ameliorate the disease, send the disease into remission, maintain the disease in remission or cure the disease. Treating as employed herein includes administration of a medicament according to the present disclosure for treatment or prophylaxis.

Treatment or therapy may be employed prophylactically.

Therapeutically effective amount as employed herein is an amount in the range which generates a desirable physiological effect, whilst minimising side effects.

Disease modifying therapy as employed herein refers to therapy that allows the immune system to reset itself and rebalance, thereby performing more normally after treatment.

The DHODH inhibitor of the disclosure or formulation comprising the same may be administered at a dose in the range of 1 mg to 400 mg per day, such as 10 mg to 400 mg per day, 50 mg to 400 mg per day, 100 mg to 400 mg per day, 150 mg to 400 mg per day, 200 mg to 400 mg per day, 250 mg to 400 mg per day, 300 mg to 400 mg per day, or 350 mg to 400 mg per day.

In particular, a dose in the range of 100 mg to 400 mg per day is administered.

Thus, the daily dose may be for example 10 mg 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg 320 mg, 330 mg 340 mg, 350 mg, 360 mg 370 mg, 380 mg, 390 mg or 400 mg.

In one embodiment the treatment is administered daily, for example once or twice daily.

In one embodiment the treatment is once daily.

In one embodiment ASLAN003 is administered orally, for example as a tablet or capsule or caplet.

Co-morbidity as employed herein refers to where the patient is suffering from a second or underlying health condition.

Combination therapy (comprising further therapy) as employed herein wherein two or more treatment regimens are employed, in particularly employed concomitantly. The treatments may be separate formulations or co-formulated. They may be administered at the same time or different times. However, the pharmacological effect of the treatments will co-exist in the patient. Further therapy as employed herein refers to a therapy in addition to the DHODH inhibitor.

Such a further therapy may be an anti-inflammatory agent, which includes but is not limited to, a non-steroidal anti-inflammatory agent (NSAID), a disease modifying anti-rheumatic drug (DMARD), a statin (including HMG-CoA reductase inhibitors such as simvastatin), a biological agent (biologicals), a steroid, an immunosuppressive agent, a salicylate and/or a microbicidal agent.

Non-steroidal anti-inflammatory agents include anti-metabolite agents (such as methotrexate) and anti-inflammatory gold agents (including gold sodium thiomalate, aurothiomalate or gold salts, such as auranofin). Biologicals include anti-TNF agents (including adalimumab, etanercept, infliximab, anti-IL-1 reagents, anti-IL-6 reagents, anti-CD20 agents, anti-B cell reagents (such as rituximab), anti-T cell reagents (anti-CD4 antibodies), anti-IL-15 reagents, anti-CLTA4 reagents, anti-RAGE reagents), antibodies, soluble receptors, receptor binding proteins, cytokine binding proteins, mutant proteins with altered or attenuated functions, RNAi, polynucleotide aptamers, antisense oligonucleotides or omega 3 fatty acids. Steroids (also known as corticosteroids) include cortisone, prednisolone or dexamethasone may also be employed in a combination therapy.

Immunosuppressive agents for use in a combination therapy according to the present disclosure include cyclosporin, FK506, rapamycin, mycophenolic acid. Salicylates for use in said combination therapy include aspirin, sodium salicylate, choline salicylate and magnesium salicylate. Microbicidal agents include quinine and chloroquine.

Anti-inflammatory as employed herein refers to a moiety that reduced inflammation, for a non-steroidal anti-inflammatory, steroids and the like.

In one embodiment, the combination therapy comprises an anti-CD20 agent or a biosimilar thereof, for example Rituxan (rituximab), a Rituximab biosimilar, Gazyva, Kesimpta, Ocrevus (ocrelizumab), Ruxience, Truxima, Zevalin, Arzerra, AcellBia, HLX01, Reditux, Ritucad or Zytux.

In one embodiment, the combination therapy comprises a treatment independently selected from corticosteroids (for example oral prednisone and intravenous methylprednisolone), plasma exchange (plasmapheresis), interferon beta medications, glatiramer acetate, fingolimod, dimethyl fumarate, diroximel fumarate, teriflunomide, siponimod, cladribine, ocrelizumab, natalizumab and alemtuzumab.

In one embodiment, the combination therapy comprises a treatment to ease or reduce the symptoms of multiple sclerosis, for example a muscle relaxant (such as baclofen, tizanidine and cyclobenzaprine), a medication to reduce fatigue (such as amantadine, modafinil, methylphenidate, or a medication to increase walking speed (such as dalfampridine).

In one embodiment the combination therapy comprises cannabis or a derivative thereof, for example cannabis oil.

In one embodiment the combination therapy comprises a second DHODH inhibitor. In one embodiment, the further therapy comprises teriflunomide. In one embodiment the further therapy comprises vidofludimus. In one embodiment the combination therapy does not comprise a second DHODH inhibitor. In embodiment the combination therapy does not comprise teriflunomide and/or vidofludimus.

In one embodiment the combination therapy comprises a disease modifying therapy, for example selected from alemtuzumab, avonex, betaferon, cladribine, daclizumab, dimethyl fumerate, extavia, fingolimod, glatiramer acetate, natalizumab, ocrelizumab, plegridy, rebif, siponimod and combinations of two or more of the same.

In one embodiment, the further therapy comprises interferon beta (IFN-β) such as interferon beta-1a or interferon beta-1b. Hence, in one embodiment, the further therapy comprises interferon beta-1a. In an alternative embodiment, the further therapy comprises interferon beta-1b.

In one embodiment, the combination therapy comprises a Bruton's Tyrosine Kinase (BTK) inhibitor, for example Ibrutinib, Acalabrutinib, Zanubrutinib, Evobrutinib, ABBV-105, Fenebrutinib, GS-4059, Spebrutinib and/or HM71224.

In one embodiment, the further therapy comprises glatiramer acetate. In one embodiment, the further therapy comprises natalizumab. In one embodiment, the further therapy comprises mitoxantrone. In one embodiment, the further therapy comprises fingolimod. In one embodiment the further therapy comprises Siponimod. In one embodiment, the further therapy comprises dimethyl fumarate. In one embodiment, the further therapy comprises alemtuzumab. In one embodiment, the further therapy comprises cyclophosphamide. In one embodiment, the further therapy comprises cladribine. In one embodiment, the further therapy comprises ocrelizumab. In one embodiment, the further therapy comprises dimethyl fumarate. In one embodiment, the further therapy comprises daclizumab. In on embodiment, the further therapy comprises azathioprine. In one embodiment, the further therapy comprises methotrexate. In an alternative embodiment, the further therapy does not comprise methotrexate. In one embodiment, the further therapy comprises lacquinimod.

Comprising in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present application claims from priority from Singaporean applications serial number 10202010254Q filed 15 Oct. 2020, and 10202012817S filed 21 Dec. 2020, both incorporated herein by reference. These documents may be used as the basis for corrections.

The background contains technical information and may be used as basis for amendment.

The invention will now be described with reference to the following examples, which are merely illustrative and should not be construed as limiting the scope of the present invention.

### EXAMPLES

### Example 1 In Vivo Study of ASLAN003 in Multiple Sclerosis EAE Model

Experimental autoimmune encephalomyelitis (EAE) is a well-studied animal model of demyelinating diseases, such as human multiple sclerosis (MS). EAE is induced by injecting susceptible animals with purified myelin components, central nervous system (CNS) extract, or synthesized specific peptides emulsified in an adjuvant. The peptides are for example derived from myelin basic protein (MBP), myelin oligodendrocyte glycoprotein (MOG) or proteolipid protein (PLP).

To investigate the effect of ASLAN003 on multiple sclerosis, ASLAN003 was administered orally to the EAE model at doses of 1 mg/kg, 3 mg/kg, 10 mg/kg or 15 mg/kg per day, starting from Day 8 after disease induction. A vehicle control was included.

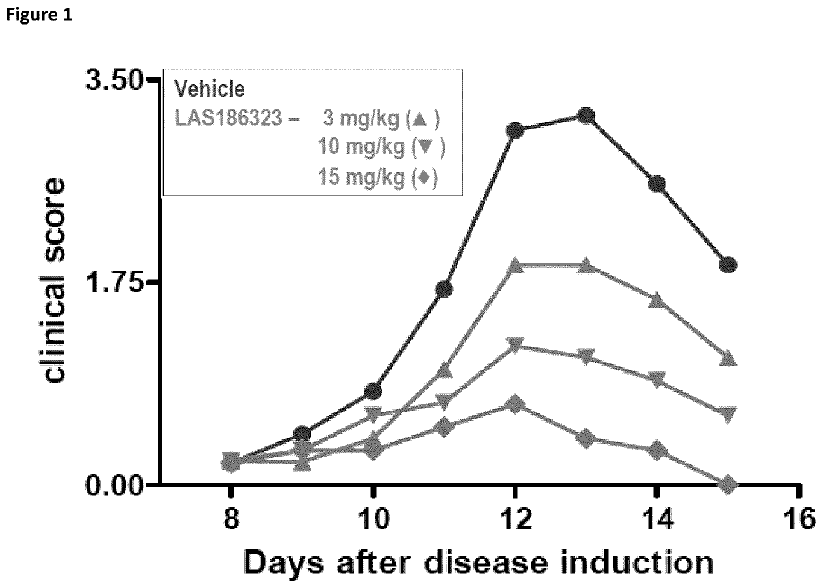

The results are shown in FIG. 1. As can be seen, ASLAN003 (LAS186323) results in a significantly reduced clinical score compared to the vehicle control. In addition, ASLAN003 appears to halt disease progress in a dose-dependent manner.

Thus, the data provides strong evidence of the potential of ASLAN003 for treating autoimmune diseases, such as multiple sclerosis.

### Example 2 In Vivo Study of ASLAN003 in EAE Model in Comparison with Positive Control T

### 1. Experimental Protocol

### 1.1. Induction of EAE and Clinical Score

For the induction of EAE, guinea pig myelin basic protein (MBP) (Sigma, M2295) suspended in 0.9% saline solution at a concentration of 2 mg/ml was used. The solution was emulsified with an equal volume of Freund's complete adjuvant (Sigma, F5881) containing 4 mg/ml of heat-inactivated Mycobacterium tuberculosis H37Ra (Difeo Laboratories, Ref. 231141). Male Lewis rats were immunized with 0.1 ml of emulsion by subcutaneous injection into the right and left hind footpads with the emulsion containing MBP at 100 μg/rat.

A group of naive animals in which the disease was not induced was included in each EAE experiment (n=5) as a healthy control for comparative purposes.

Individual animals were examined daily for clinical signs of neurological deficits scored on a 0 to 5 scale, as follows:

TABLE 1
Scoring system for neurological deficits

| | |
|---|---|
| 0 | Normal |
| 0.5 | Distal half of the tail flaccid |
| 1 | Completely flaccid tail |
| 1.5 | Partially paralyzed hind paws. Animal strong enough |
| | to drag inside the cage. |
| 2 | Lower part of the body totally paralyzed, but still |
| | able to drag inside the cage. |
| 2.5 | Lower part of the body totally paralyzed and upper |
| | part partially paralyzed. Important |
| | difficulties to drag. The animal gets tired easily |
| | but is alert and responds to stimuli. |
| 3 | Complete paralysis of lower and upper parts of the |
| | body. Unable to move (still may circle). |
| 4 | Really weak animal, without response capability. |
| | Fast breathing. Coldness. |
| 5 | Death |

Clinical signs of the disease were observed on day 8-9 after induction. When around 10% of the animals showed clinical symptoms, they were randomized into different groups (n=7-8) and treatment was initiated.

As disease progressed, animals with a score of 4 were sacrificed if disability indicated little chance of recovery, in accordance with animal welfare standards. Mortality due to sacrifice or spontaneous EAE-related death was recorded as a 5 on the given day. This death score continued to be included in the clinical assessment, but no body weight measurements could be carried forward.

### 1.2. Compounds

LAS 186323 (ASLAN003), positive control T and positive control F were freshly prepared every day and suspended in 0.5% Methylcellulose and 0.1% Tween 80 in water, at the specified doses and administered by oral gavage in a volume of 10 ml/kg.

### 1.3. Haematological Cell Count

At the end of the assay, 24 h after last administration, blood samples from the retro-orbital plexus were obtained from all animals under light anaesthesia. Samples were used to determine complete blood cell count (XT-2000i/XT-1800i, Sysmex Corporation). Absolute values were reported for all cell types.

### 1.4. Histology of the CNS

At the end of the experiment, animals were killed by exsanguination and necropsies were performed in a blind manner. The spinal cords (CNS; spinal cord, medulla oblonga, cerebellum) of EAE animals were obtained, fixed in 4% formalin in PBS, and embedded in paraffin. Six μm-thickness sections were prepared and then stained with hematoxylin and eosin. The histological score was evaluated as follows:
0: no lesions;
1: solitary lesions with cell infiltrates of low cellular density in the entire section;
2: a few lesions with moderate cell infiltration in each of a few fields;
3: many lesions in almost all fields with massive cell infiltration and accompanying edema (Kataoka, 2005).

### 2. Results

### 2.1. Clinical Score

Evaluation of disability was performed on a daily basis and a clinical score given to each animal, as described. The area under the curve (AUC) of the clinical score was generated for each animal from the data recorded along the experiment. The percentage of inhibition of the AUC was calculated for every animal versus the mean of the AUC of the vehicle group. The mean percentage of inhibition per treatment and dose was calculated.

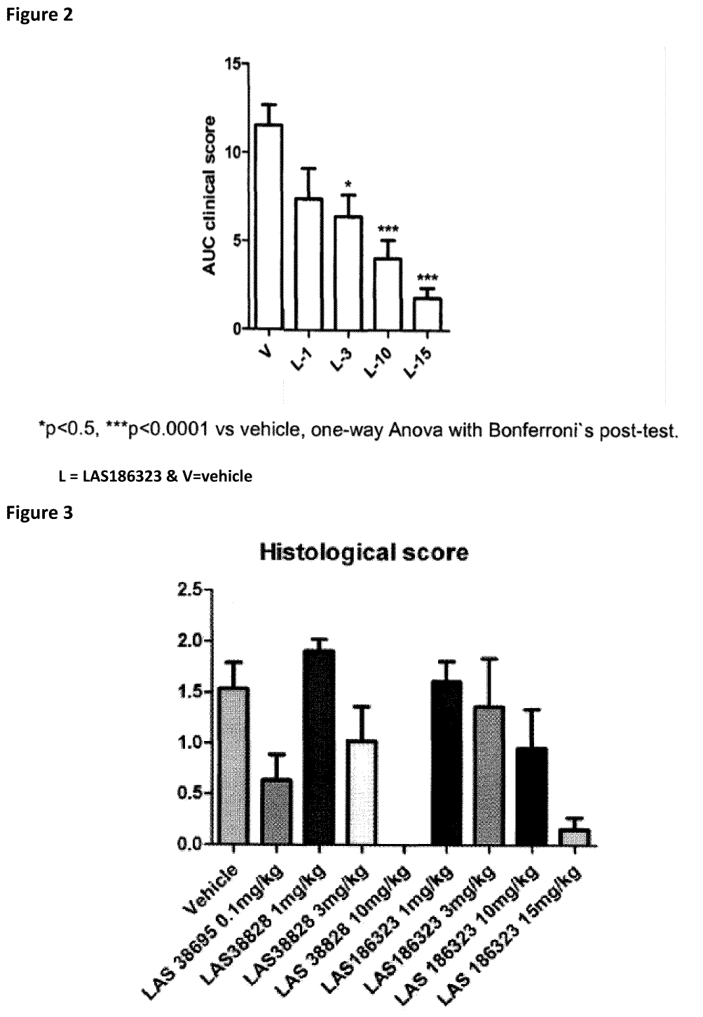

FIG. 2 shows the mean of the clinical score AUC for each treatment group; vehicle (V), and LAS186323 (ASLAN003) (L). The numbers after the initials correspond to the administered oral daily dose of the compound in mg/kg.

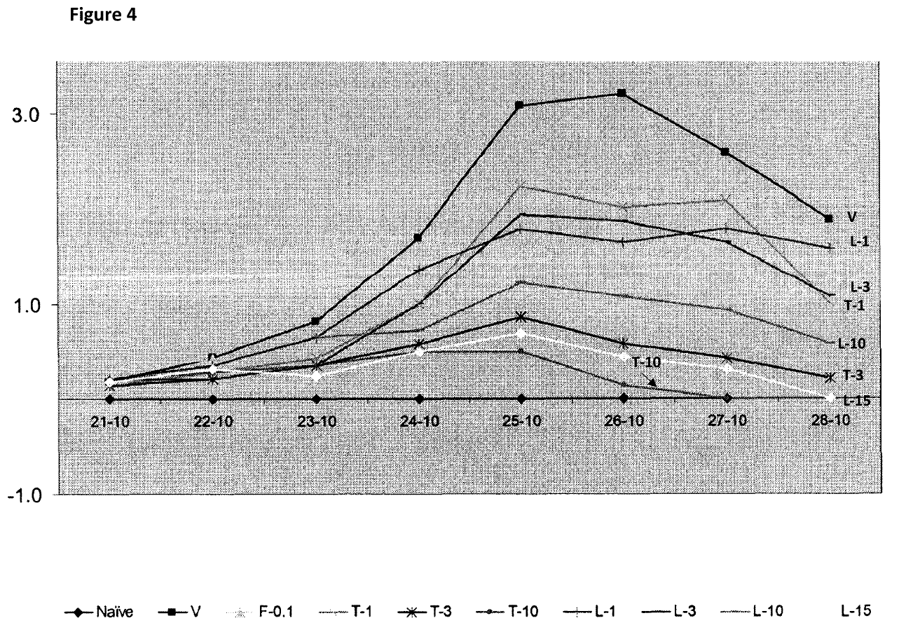

FIG. 4 shows the evolution of the disease with time. Each point represents the mean of the clinical score (Y-axis) at that particular day of treatment (X-axis). Vehicle (V), positive control (F), positive control (T) and LAS186323 (L). The numbers after the initials correspond to the administered oral daily dose of the compound in mg/kg.

Table 2 below shows the percentage inhibition of the clinical score, which is the measure of clinical efficacy.

TABLE 2
% inhibition of clinical score

| | Dose | % AUC inhibition |
|---|---|---|
| Compound | (mg/kg) | Mean (SEM) |
| LAS38695 | 0.1 | 73 (9.4) |
| (Positive control F) | | |
| LAS186323 | 1 | 41.1 (13.34) |
| (ASLAN003) | 3 | 44.4 (10.79) |
| | 10 | 65.1 (9.02) |
| | 15 | 84.4 (4.94) |
| LAS38828 | 1 | 33.5 (4.44) |
| (Positive control T) | 3 | 78.3 (7.66) |
| | 10 | 90.2 (4.29) |

Although a proper ED₅₀ cannot be calculated for positive control T, an estimated value of 1.5 mg/kg has been calculated. The ED₅₀ for ASLAN003, calculated by linear regression is 2.7 mg/kg/day.

### 2.2. CNS Histology Evaluation

FIG. 3 depicts the CNS histological score. A correspondence between improvement of the clinical score and the histological score was observed. A good dose-response was obtained with both positive control T and ASLAN003, with a more abrupt slope observed for the former compound. At the highest doses tested, animals treated with positive control T or ASLAN003 showed a profound improvement in the microscopic CNS lesions compared to vehicle-treated animals.

### 2.3. Haematological Cell Count

Blood samples were collected 24 hours after last administration and haematological cell count was performed as described above. FIG. 5 shows the absolute cell count numbers in cells×10³/μl per treatment group. A significant effect of positive control F on the total lymphocyte count was observed as expected, due to its mechanism of action. A reduction in the number of neutrophils was observed in animals treated with positive control T at 10 mg/kg and ASLAN003 at 3 and 15 mg/kg, but not at 10 mg/kg. However, no statistically significant difference versus vehicle was observed. A mild dose-response on red blood cell count (RBC) was observed with positive control T.

### 3. Conclusions

Based on the above results, ASLAN003 appears to inhibit disease progression in the EAE Lewis rat model in a dose dependent manner when administered once a day by oral route to animals with established disease. Improvement of the external neurological symptoms corresponds to a decrease in the microscopic damage in the CNS. Treatment with ASLAN003 did not have a significant effect on blood cell count.

### Example 3—In Vivo Study of ASLAN003 vs Positive Control T in Rheumatoid Arthritis (RA) AIA Model

Adjuvant-induced arthritis (AIA) is an experimental model of rheumatoid arthritis (RA) induced in rats by the intraplanar injection of complete Freund's Adjuvant (CFA). The disease progresses with a strong inflammation on the injected paw. The systemic inflammation and immunological alterations caused by the CFA translates into the inflammation of the contralateral paw from day 7 to day 10 post-induction.

The ability of the AIA model to predict efficacy in the clinic is well established.

Hence, to investigate the effect of ASLAN003 on RA, ASLAN003 was administered to AIA rat models.

### 1. Experimental Protocol

### 1.1. Test Compounds

ASLAN003 was freshly prepared every day as a suspension in 0.5% Methylcellulose and 0.1% Tween in water, at the specified doses and administered by oral gavage in a volume of 10 ml/kg.

### 1.2 Animals

Male Wistar rats with a body weight of 175-200 g were used.

### 1.3 Arthritis Induction

A suspension of 75 mg of a desiccated extract of Mycobacterium tuberculosis H37 RA (Difco, #231141) was prepared by adding 15 ml of paraffin oil (Merck, #7162) and some drops of distilled water. The concentration of Mycobacterium in the CFA was 5 mg/ml. The suspension was sonicated for 10 mins and maintained in a shaker during the whole process. Rats were anesthetised and 0.1 ml of the CFA suspension was injected intraplantarly into their hind left paws.

10 days after disease induction, the inflammation of the hind paws of every rat was measured by plethysmometry (Ugo Basile, #7140). Inflammation was expressed in ml. On Day 1, which is the first day of administration, rats (n=6-7) having similar volume in both paws were assigned to a treatment group.

Rats were administered the CFA suspension every day in the morning for 10 consecutive days. Body weight was measured every day and paw volumes every other day. On day 11 post-treatment (24 hrs after last administration), the rats were anesthetised, blood samples were collected from their retroorbital plexus to determine cell counts, and then sacrificed. Hind paws were excised to perform X-ray analysis and a score of radiological damage was performed by a scientist unaware in a blind manner.

The radiological score is a composite of 5 different parameters evaluated from the X-ray images of every individual paw. The 5 parameters are: bone demineralisation, periostitis, narrowing of the joint space, cystic degeneration, and inflammation of the soft tissues. For each parameter, a value of 0-4 was assigned, which is proportional to the severity observed. The sum of the scores of the 5 parameters provides the radiological score for each animal.

### 1.4 Calculations

For each animal, inflammation was measured as the area under curve (AUC) of the right paw volume. AUC was calculated by plotting paw inflammation in ml vs time in days. The mean value of the vehicle-treated group was also obtained.

The efficacy of a treatment was calculated as the % inhibition of each animal vs the mean of the vehicle. A mean for every group of treatment was calculated. In the case where more than one experiment was performed for a given compound and dose, the mean±standard error of the mean (SEM) was calculated.

The effect of a treatment on the inhibition of the radiological score was measured by calculating the % inhibition of the radiological score of every animal belonging to a treatment group vs the mean of the vehicle treated group. In the case where several experiments were conducted, the values provided are the mean of the individual experiments and the SEM.

### 2. Results

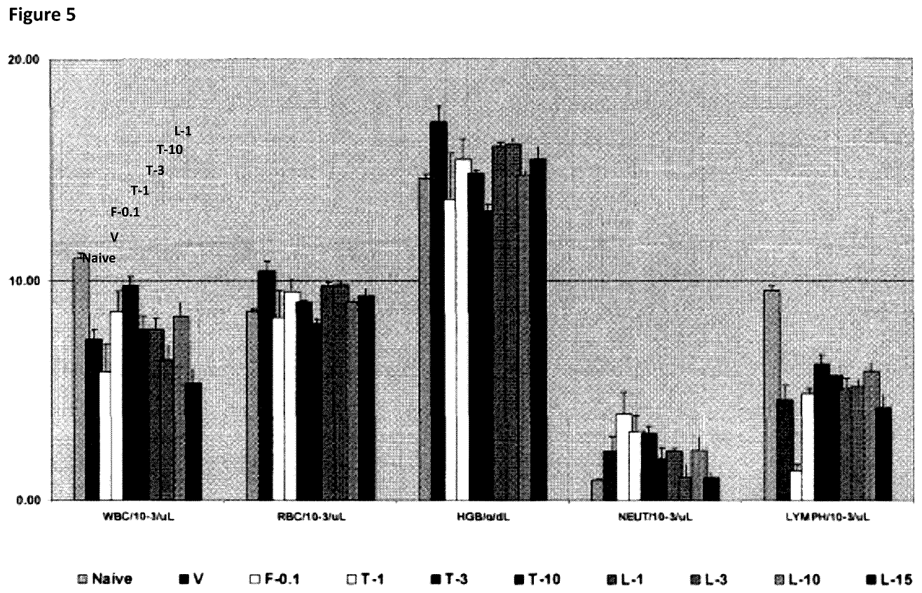

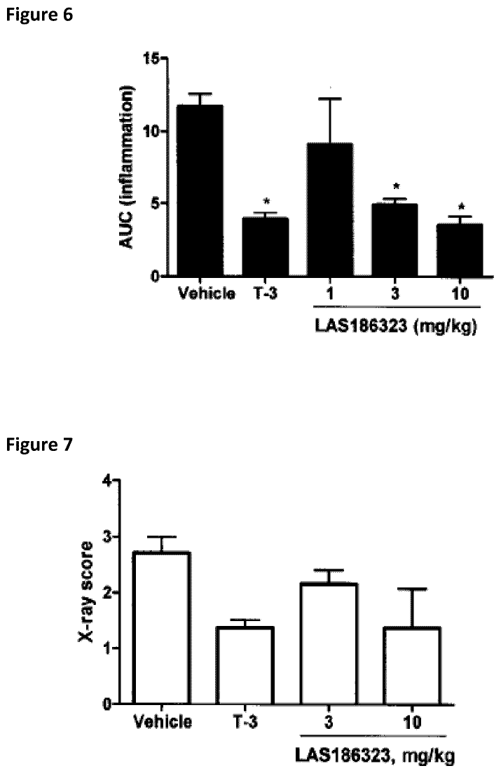

FIG. 6 shows the AUC of the paw inflammation and Table 3 below shows the numerical values of the AUC was well as the % inhibition of the AUC values expressed as the mean±SEM values from 2 to 5 independent experiments.

TABLE 3
AUC and % inhibition for ASLAN003 in AIA model

| | | Positive Control T | ASLAN003 | | |
|---|---|---|---|---|---|
| | Vehicle | 3 mg/kg | 1 mg/kg | 3 mg/kg | 10 mg/kg |
| AUC | 11.73 ± 0.85 | 3.90 ± 0.47* | 9.11 ± 3.14 | 4.91 ± 0.43* | 3.49 ± 0.63* |
| % | — | 62.46 ± 4.9 | 32.76 ± 22.8 | 53.09 ± 2.4 | 64.49 ± 4.6 |
| Inhibition | | | | | |

One-way ANOVA with Bonferroni post test,
*p < 0.05 vs vehicle

ASLAN003 at 3 and 10 mg/kg significantly reduced right paw swelling compared to vehicle-treated rats. The lowest AUC values, indicating higher anti-inflammatory effect, was achieved in the groups treated with positive control T and ASLAN003 at 10 mg/kg.

The mean % reduction in paw volume from 2 to 5 independent experiments was used to calculate an ED₅₀ value of 4.6 mg/kg for ASLAN003.

Table 4 below shows the white blood cell (WBC) and platelet counts for the different treatment groups. The comparison of the blood cell counts among the different treatment groups shows that the induction of arthritis causes a significant increase in both WBC and platelets (vehicle vs naïve rats). In arthritic rats treated with positive control Tat 3 mg/kg or ASLAN003 at 10 mg/kg, a significant reduction of the WBC numbers vs vehicle was observed. At the same dose, ASLAN003 also significantly decreased the number of platelets vs vehicle-treated rats.

TABLE 4
WBC and platelet counts in AIA model

| | Dose | White blood | |
|---|---|---|---|
| Treatment | (mg/kg) | cells ×10³/μl | Platelets ×10³/μl |
| Naïve (healthy) | | 9.2 ± 0.3 | 667 ± 14.5 |
| Vehicle | | 17.4 ± 1.5^{#} | 1394 ± 44.2^{#} |
| ASLAN003 | 1 | 20.6 ± 0^{#} | 1418 ± 180^{#} |
| | 3 | 16.2 ± 0.86^{#} | 1180 ± 45.9^{#} |
| | 10 | 5.4 ± 0.8* | 402 ± 137* |
| Positive Control T | 3 | 12.58 ± 1.2* | 1110 ± 106^{#} |

^{#}p < 0.05 vs naïve,
*p < 0.05 vs vehicle

The radiological score of the paw of arthritic rats treated with ASLAN003 at the highest doses (3 mg/kg and 10 mg/kg) or positive control T is shown in FIG. 7. The numerical values are shown in Table 5 below. The results are expressed as the mean±SEM of 2 to 3 independent experiments.

TABLE 5
X-ray scores in AIA model

X-ray score

| | Positive control T | ASLAN003 | ASLAN003 |
|---|---|---|---|
| Vehicle | 3 mg/kg | 3 mg/kg | 10 mg/kg |
| 2.71 ± 0.29 | 1.38 ± 0.14 | 2.16 ± 0.25 | 1.38 ± 0.72 |

The results indicate that although the mean score for both positive control T at 3 mg/kg and ASLAN003 at 10 mg/kg was lower than the score for the vehicle-treated rats, the difference was not statistically different.

### 3. Conclusions

ASLAN003 administered once daily orally shows an anti-inflammatory effect in the paws of rats with established arthritis. A dose-dependent decrease in the number of WBC and platelets in peripheral blood was also observed which reaches statistical significance at the highest dose tested. ASLAN003 improves the radiological score of the paw, indicating disease-modifying potential.

In conclusion, this study demonstrates the potential of ASLAN003 as an effective treatment for RA.

### Example 4—In Vivo Study of ASLAN003 vs Positive Control V in Dextran Sulfate Sodium (DSS) Induced Inflammatory Bowel Disease (IBD) Mouse Model

### 1. Experimental Protocol

### 1.1 Animals

Male C57BL/6 mice aged 6-7 weeks were procured from Shanghai Lingchang Bio Tech Co. Ltd, China.

### 1.2 Treatment Schedule, Sample Collection and Parameters

Animals were randomized into groups based on the body weight:
Group 01: Purified water, ad libitum, Day 1-8; Vehicle, 5 mL/kg, P0, QD, Day 0-9, n=6
Group 02: 3% DSS in water, ad libitum, Day 1-8; Vehicle, 5 mL/kg, P0, QD, Day 0-9, n=8
Group 03: 3% DSS in water, ad libitum, Day 1-8; positive control V, 20 mg/kg, P0, QD, Day 0-9, n=8
Group 04: 3% DSS in water, ad libitum, Day 1-8; ASLAN003, low dose, P0, QD, Day 0-9, n=8a
Group 05: 3% DSS in water, ad libitum, Day 1-8; ASLAN003, high dose, P0, QD, Day 0-9, n=8
Abbreviations: QD=Once daily; PO=Oral gavage.
Study duration: 3 weeks of acclimation phase, and 10 days of treatment phase starting from Day 0 (Day 0-10).
Colitis model induction: To induce acute experimental colitis, mice in Groups 02-05 were provided ad libitum access to 3% DSS in purified water for 7 days (Day 1-7), followed by purified water for 2 days (Day 9-10). The drinking water containing DSS was changed once in two days. Mice in Group 01 was kept on purified water as a control, during Day 1-7.
In-life parameters measured:
   Water and food consumption was monitored once daily during Day 0-10.
   Body weight was measured once daily during Day 0-10, with twice daily clinical monitoring.
   Stool observation as conducted once daily during Day 0-10. A disease activity index (DAI) was determined to quantify induction of colitis.
   Intestinal permeability was assessed on Days 0 and 9.
   Faeces was collected for lipocalin-2 detection on Days 0, 4, 8 and 10.
Study Termination: The mice were sacrificed on Day 10, and the following was performed on the sacrificed animals:

### Blood Collection

Mice were bled to prepare serum, and store at −60° C. to −80° C. for potential cytokine assays.

### Colon Collection

The entire colon (from caecum to anus) was collected and its length from ileocecal valve to anus was measured.
One photo of the entire colon for each mouse was taken.
The colon was opened along the mesenteric border, rinsed with saline, blotted dry and its weight was measured.
The colon was processed as ‘spiral Swiss Rolls’, fixed in 10% neutral-buffered formalin for 24-48 hours, and embedded in paraffin (one block per mouse) for hematoxylin-eosin (H&E) staining and histopathological evaluation.

### Spleen Weight

The spleen was dissected, its tissue weight measured and recorded, and then discarded.

### 2. Results

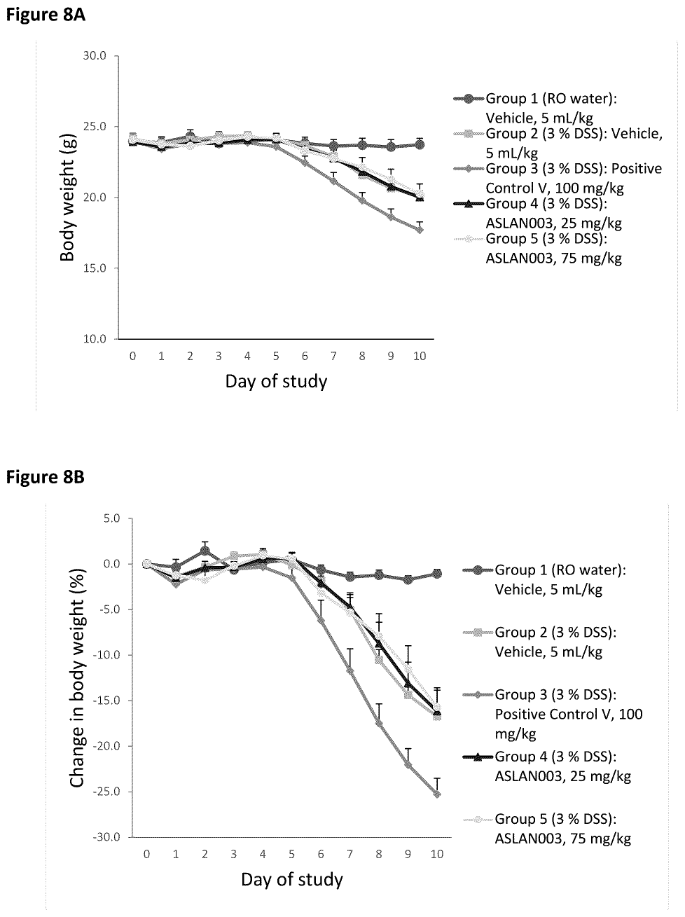

The results of the study are shown in FIGS. 8 to 13.

FIGS. 8A and 8B show the body weight measurements of the animals over the course of the study. The loss in body weight was similar for DSS induced vehicle group 2 and the two ASLAN003 treated groups 4 and 5, while the loss in body weight was most acute for positive control V treated group 3.

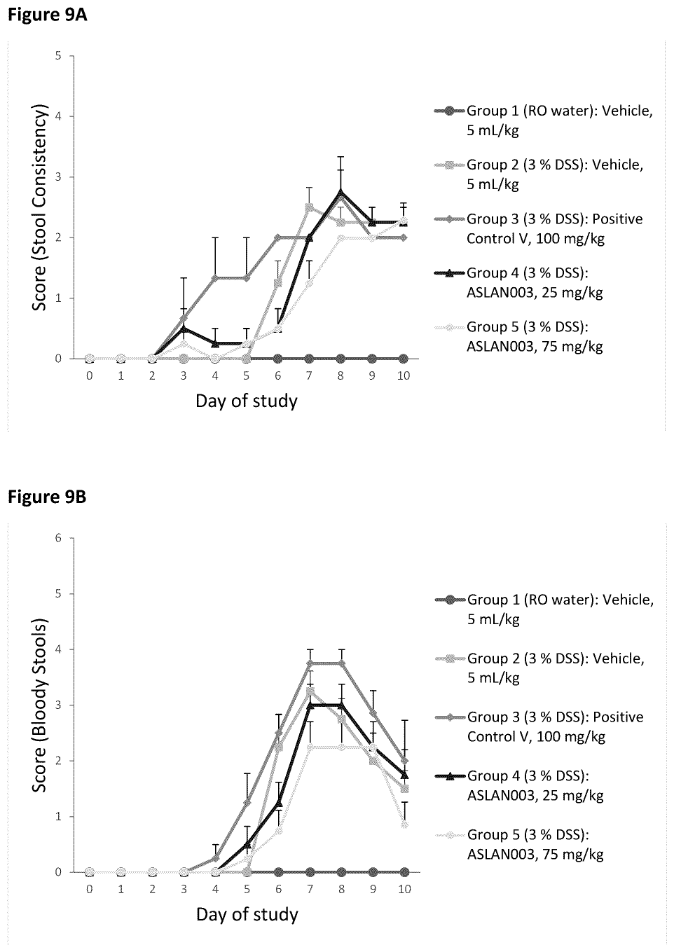

FIGS. 9A and 9B show the stool scores for the animals over the course of the study. A higher stool consistency score in FIG. 9A indicates a lower stool consistency, whilst a higher bloody stool score in FIG. 9B indicates more bloody stools observed.

As can be seen, positive control V treated group 3 had a lower overall stool consistency compared to the other DSS induced animals during the earlier part of the study, before finally achieving the best stool consistency on Day 10. The ASLAN003 75 mg/kg group 5 had the best overall stool consistency from Days 2 to 9, before ending the study with a similar stool consistency score as Vehicle group 2 and ASLAN003 25 mg/kg group 4 on Day 10.

In terms of bloody stools, the results suggest that the ASLAN003, 75 mg/kg treated group 5 had the lowest incidence of bloody stools over the course of the study. Interestingly, the positive control V treated group 3 had the highest incidence of bloody stools, appearing to even exceed that of vehicle treated group 2.

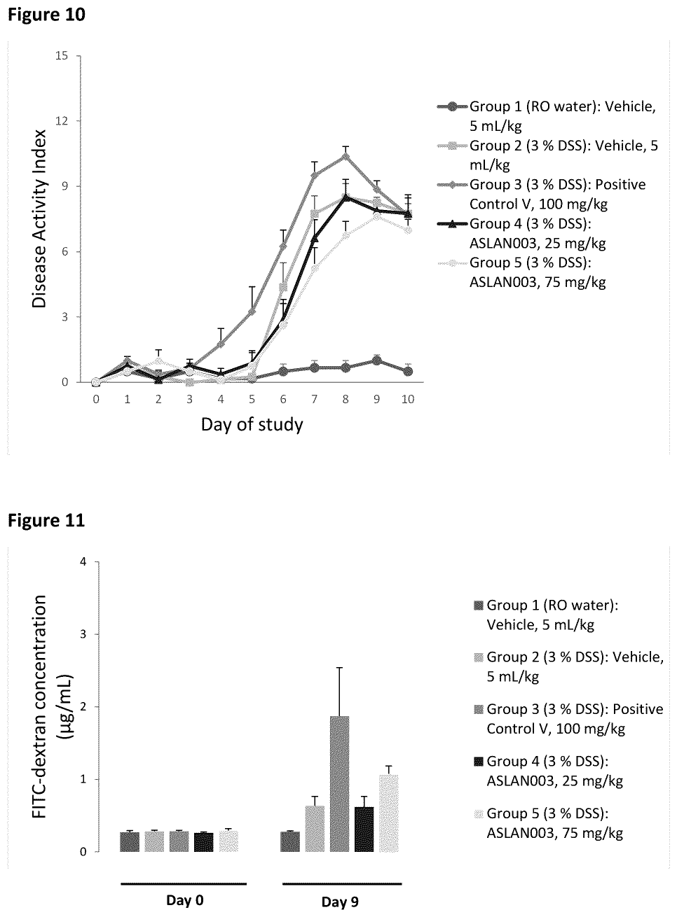

FIG. 10 shows the Disease Activity Index (DAI) for the animals over the course of the study. The DAI is a scoring mechanism used to assess colitis in patients and is a combined score based on weight loss, stool consistency and bleeding. The results indicate that the DAI increased the most in positive control V treated group 3 before eventually dropping to similar level to vehicle group 2 and ASLAN003, 25 mg/kg group 4 by Day 10 of the study. In addition, the results appear to show a lower overall increase in DAI over the 10 days for ASLAN003, 75 mg/kg group 5, as well as a lower DAI score at Day 10 compared to all the other treatment groups.

FIG. 11 shows the intestinal permeability (FITC-dextran concentration) results for the animals on Days 0 and 9 of the study. The ASLAN003, 25 mg/kg treated group 4 and vehicle group 2 appear to have similar FITC-dextran concentrations measurements on Day 9. Surprisingly positive control V treated group 3 had a noticeably higher measurement than all the other treatment groups on Day 9, suggesting that intestinal permeability of the animals in this group was significantly altered compared to the control group 1.

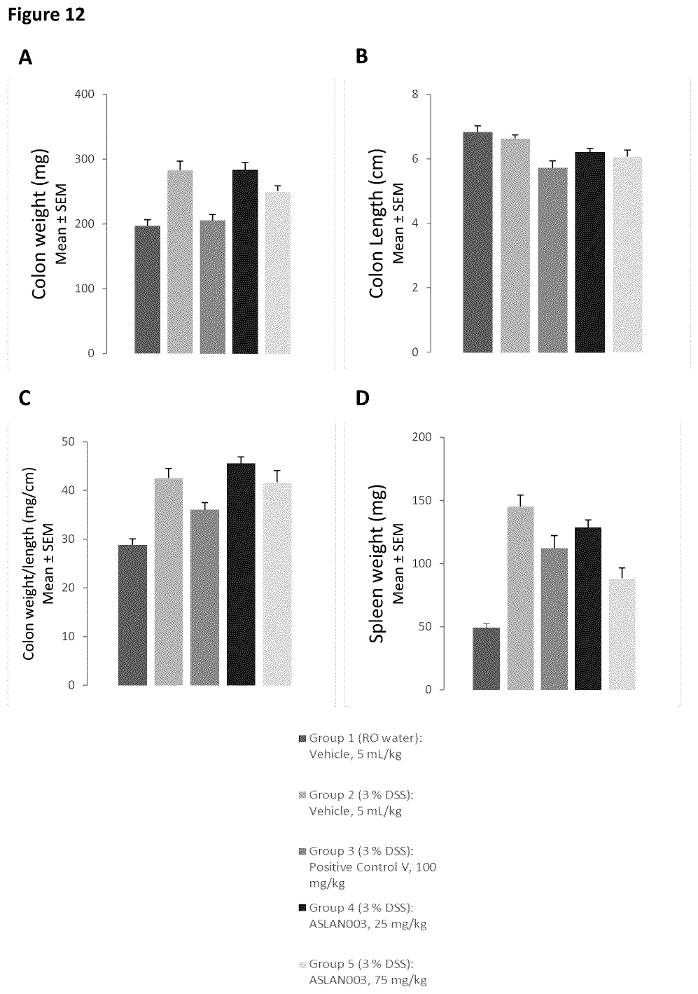

FIG. 12 shows the colon and spleen measurement results for the different treatment groups at the end of the study. When both colon weight and length are taken into consideration as in FIG. 12C, the results appear to indicate that the colon/length measurements are fairly similar for the Vehicle group 2 and ASLAN003 treated groups 4 and 5. The positive control V treated group 3 had a colon/length measurement that was more similar to the control group 1.

FIG. 12D seems to suggest that ASLAN003, 75 mg/kg treated group 5 had a spleen weight that was most similar to the control group 1, with the other treatment groups having comparable spleen weights to each other.

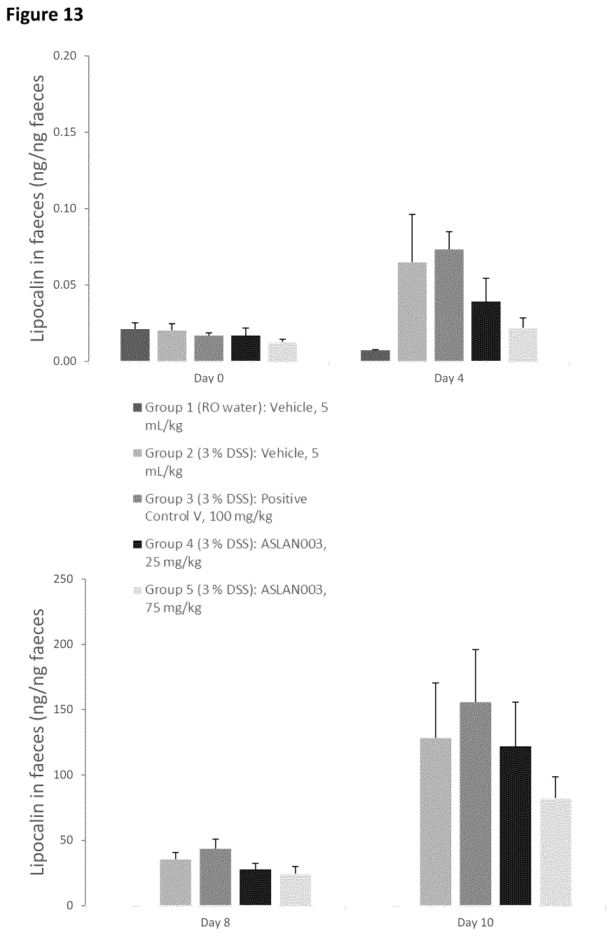

FIG. 13 shows the lipocalin in faeces data for the animals over the course of the study. Lipocalin is a sensitive and non-invasive biomarker of intestinal inflammation. The results indicate that the ASLAN003 treated groups 4 and 5 had less intestinal inflammation than vehicle treated group 2. Conversely, positive control V treated group 3 appeared to show a greater degree of intestinal inflammation compared to vehicle treated group 2.

In summary, the results indicate that:
1. The ASLAN003 treated groups 4 and 5 showed improvements in DAI, stool consistency, bloody stools, and lipocalin in faeces compared to the DSS induced vehicle group 2.
2. In particular, the ASLAN003, 75 mg/kg treated group 5 had the lowest overall increase in DAI, the best overall stool consistency, the lowest incidence of bloody stools, and lowest lipocalin in faeces compared to the other DSS induced groups. This treatment group also had a spleen weight that was most similar to that of control group 1.
3. The positive control V treated group 3 had the greatest loss in body weight, the greatest overall increase in DAI, the lowest overall stool consistency, highest incidence of bloody stools, highest intestinal permeability, and highest lipocalin in faeces compared to the other DSS induced groups. This treatment group also had a colon/length measurement that was most similar to that of control group 1.

These results demonstrate that the ASLAN003 treated animals showed improvements in a range of different parameters compared to the controls, suggesting that ASLAN003 has a strong potential to be useful in the treatment of inflammatory bowel disease. The results also indicate that the use of ASLAN003, in particular at the higher 75 m/kg concentration, resulted in greater improvements to the animals compared to the controls. Unexpectedly, the results further suggest that the positive control V treated animals had less desirable outcomes compared to vehicle group 2 for some of the parameters tested.

1-18. (canceled)

19. A method of treating an autoimmune disease comprising administering orally once or twice daily a therapeutically effective amount of a pharmaceutical formulation comprising DHODH inhibitor 2-(3,5-difluoro-3′methoxybiphenyl-4-ylamino)nicotinic acid or a pharmaceutically acceptable salt thereof, wherein the autoimmune disease is selected from the group comprising relapsing-remitting multiple sclerosis, and inflammatory bowel disease; and wherein the DHODH inhibitor is administered at a dose in the range of 10 mg to 400 mg per day, with the proviso that the treatment is not used in combination with methotrexate.

20. The method according to claim 19, wherein the autoimmune disease is relapsing-remitting multiple sclerosis.

21. The method according to claim 19, wherein the autoimmune disease is inflammatory bowel disease.

22. The method according to claim 21, wherein the inflammatory bowel disease is selected from Coeliac disease, Crohn's disease, and Ulcerative colitis.

23. The method according to claim 19, wherein the autoimmune disease is characterised by aberrant T cell and/or B cell activation.

24. The method according to claim 19, wherein the autoimmune disease is severe in that is not controlled by standard of care medicaments/treatments.

25. The method according to claim 19, wherein the DHODH inhibitor is employed as monotherapy.

26. The method according to claim 19, wherein the DHODH inhibitor is employed in a combination therapy.

27. The method according to claim 26, wherein the combination therapy comprises a treatment independently selected from:
i. corticosteroids (for example oral prednisone and intravenous methylprednisolone), plasma exchange (plasmapheresis), interferon beta medications, glatiramer acetate, fingolimod, dimethyl fumarate, diroximel fumarate, teriflunomide, siponimod, cladribine, ocrelizumab, natalizumab, an anti-CD20 agent or biosimilar thereof, such as rituximab, alemtuzumab, and a Bruton's Tyrosine Kinase (BTK) inhibitor;
ii. a treatment to ease or reduce the symptoms of multiple sclerosis, for example a muscle relaxant (such as baclofen, tizanidine and cyclobenzaprine), a medication to reduce fatigue (such as amantadine, modafinil, and methylphenidate), and a medication to increase walking speed (such as dalfampridine);
iii. an antidepressant, for example duloxetine, and a tricyclic antidepressant, such as clomipramine;
iv. interferon beta (IFN-β), such as interferon beta-1a or interferon beta-1b;
v. an anti-CD20 agent or a biosimilar thereof, for example Rituxan (rituximab), a Rituximab biosimilar, Gazyva, Kesimpta, Ocrevus (ocrelizumab), Ruxience, Truxima, Zevalin, Arzerra, AcellBia, HLX01, Reditux, Ritucad and Zytux; and
vi. a Bruton's Tyrosine Kinase (BTK) inhibitor, for example Ibrutinib, Acalabrutinib, Zanubrutinib, Evobrutinib, ABBV-105, Fenebrutinib, GS-4059, Spebrutinib and HM71224.

28. The method according to claim 26, wherein the combination therapy comprises a purine synthesis inhibitor, such as azathioprine.

29. The method according to claim 19, wherein the DHODH inhibitor is administered once daily.

30. The method according to claim 19, wherein the DHODH inhibitor is administered twice daily.

31. The method according to claim 19, wherein the DHODH inhibitor is administered at a dose in the range of 100 to 400 mg per day.

32. The method according to claim 31, wherein the DHODH inhibitor is administered as a dose of 100 mg per day.
